(19) United States
(12) Patent Application Publication
(10) Pub. No.: US 2021/0113141 A1
(43) Pub. Date: Apr. 22, 2021

(54) MEASURING DEVICE AND SYSTEM

(71) Applicants: Takashi IKEDA, Ishikawa (JP); Mitsuru KIKUCHI, Ishikawa (JP); Yuko YOSHIMURA, Ishikawa (JP); Hirofumi MORISE, Tokyo (JP); Kiwamu KUDO, Tokyo (JP); Yoshihiro MISAKA, Tokyo (JP); Eiichi OKUMURA, Tokyo (JP)

(72) Inventors: Takashi IKEDA, Ishikawa (JP); Mitsuru KIKUCHI, Ishikawa (JP); Yuko YOSHIMURA, Ishikawa (JP); Hirofumi MORISE, Kanagawa (JP); Kiwamu KUDO, Ishikawa (JP); Yoshihiro MISAKA, Ishikawa (JP); Eiichi OKUMURA, Ishikawa (JP)

(73) Assignees: Ricoh Company, Ltd., Ohta-ku, Tokyo (JP); National University Corporation Kanazawa University, Kanazawa-shi, Ishikawa (JP)

(21) Appl. No.: 16/981,839

(22) PCT Filed: Mar. 14, 2019
(86) PCT No.: PCT/JP2019/010690

§ 371 Date: Sep. 17, 2020

(30) Foreign Application Priority Data
Mar. 19, 2018 (JP) 2018-051791

(51) Int. Cl. A61B 5/00

(52) U.S. Cl. CPC A61B 5/4064; A61B 5/742; A61B 5/725

(57) ABSTRACT

A measuring device includes a stimulation instructing unit and an estimating unit. The stimulation instructing unit is configured to instruct a stimulating device to generate a plurality of stimulations corresponding to at least three fiducial points of brain anatomy data, the at least three fiducial points having been defined in the brain anatomy data. The estimating unit is configured to estimate, based on sensor output signals output from sensors configured to measure a brain activity signal of a subject who is a target to be measured, parts where brain activities occur due to the plurality of stimulations.

### TECHNICAL FIELD

The present invention relates to a measuring device and a system.

### BACKGROUND ART

Conventionally done frequently in brain functional imaging using a brain function measuring device, such as magnetoencephalograpy (MEG), electroencephalography (EEG), or near-infrared spectroscopy (NIRS), is brain mapping for finding out which parts of a brain measured signals have been emitted from.

Required in brain mapping is relative positioning between: brain anatomy data (for example, an MRI image) obtained by a magnetic resonance imaging (MRI) device; and brain function information obtained by a sensor of a brain function measuring device.

Disclosed in Patent Literature 1 is a technique where several fiducial points are set on a surface of the head of a subject who is a target to be measured before brain activity signals are measured, and positioning between brain anatomy data and a sensor of a brain function measuring device is performed from positional information of these fiducial points.

### SUMMARY OF INVENTION

### Technical Problem

However, since the brain function information and the brain anatomy data are associated with each other by using the information on the positions on the surface of the head, this conventional technique has a problem that the accuracy of brain mapping is reduced when, for example, the fiducial points are displaced during the measurement.

The present invention has been made in view of the above, and an object thereof is to improve positional accuracy of brain mapping.

### Solution to Problem

According to one aspect of the present invention, a measuring device includes a stimulation instructing unit and an estimating unit. The stimulation instructing unit is configured to instruct a stimulating device to generate a plurality of stimulations corresponding to at least three fiducial points of brain anatomy data, the at least three fiducial points having been defined in the brain anatomy data. The estimating unit is configured to estimate, based on sensor output signals output from sensors configured to measure a brain activity signal of a subject who is a target to be measured, parts where brain activities occur due to the plurality of stimulations.

### Advantageous Effects of Invention

According to the present invention, since brain function information and brain anatomy data are associated with each other by using, instead of information on positions on a surface of the head of a subject, information on positions in the cerebral parenchyma of the subject; an effect of enabling significant improvement in positional accuracy of brain mapping is achieved.

### BRIEF DESCRIPTION OF DRAWINGS

FIG. 1 is a diagram illustrating an example of a system configuration of a biological function measuring and analyzing system according to an embodiment.

FIG. 2 is a diagram illustrating an example of a hardware configuration of a biometric instrument for measurement and analysis.

FIG. 3 is a diagram illustrating functions of the biometric instrument for measurement and analysis.

FIG. 4A is a flow chart schematically illustrating an example of a flow of fiducial point determination processing.

FIG. 4B is a flow chart schematically illustrating another example of the flow of the fiducial point determination processing.

FIG. 4C is a flow chart schematically illustrating yet another example of the flow of the fiducial point determination processing.

FIG. 4D is a flow chart schematically illustrating still another example of the flow of the fiducial point determination processing.

FIG. 4E is a flow chart schematically illustrating yet another example of the flow of the fiducial point determination processing.

FIG. 5A is a diagram illustrating an example of a waveform of an audible sound stimulation.

FIG. 5B is a diagram illustrating the example of the waveform of an audible sound stimulation.

FIG. 6 is a diagram illustrating an example of a waveform of a visible light stimulation.

FIG. 7 is a diagram illustrating an example of a waveform of an electric stimulation.

FIG. 8 is a diagram illustrating an example of parts where brain activities occur due to a light stimulation and a sound stimulation.

FIG. 9 is a diagram illustrating an example of pars where brain activities occur due to a light stimulation and a locomotor stimulation.

FIG. 10 is a diagram illustrating an example of parts where brain activities occur due to a light stimulation and an electric stimulation.

FIG. 11 is a diagram illustrating examples of dipole estimation techniques for active parts of a brain.

### DESCRIPTION OF EMBODIMENTS

Described hereinafter in detail by reference to the accompanying drawings are exemplary embodiments of a measuring device and a system according to the present invention.

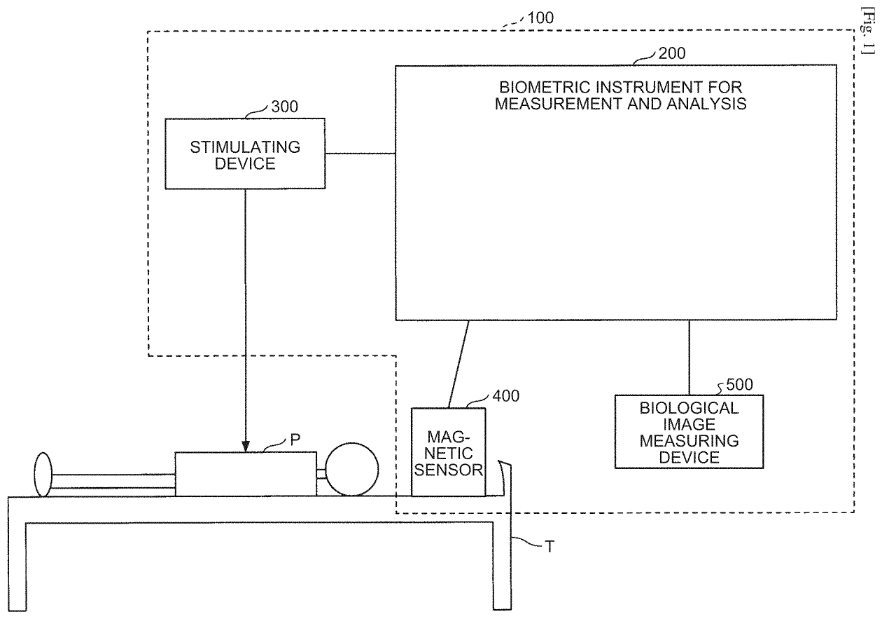

FIG. 1 is a diagram illustrating an example of a system configuration of a biological function measuring and analyzing system 100 according to an embodiment of the present invention.

The biological function measuring and analyzing system 100 according to the embodiment has: a biometric instrument for measurement and analysis 200 that is a measuring device; a stimulating device 300; a magnetic sensor 400 that forms a brain function measuring device; and a biological image measuring device 500.

In the biological function measuring and analyzing system 100, a stimulation is given by the stimulating device 300 to a subject P to cause a neural activity of the brain of the subject P to be induced, and a magnetic field generated from the neural activity is detected by the magnetic sensor 400. The magnetic sensor 400 outputs a result of the detection, to the biometric instrument for measurement and analysis 200. A signal output from the magnetic sensor 400 to the biometric instrument for measurement and analysis 200 will be referred to as a sensor output signal.

The biological image measuring device 500 is an MRI device that captures magnetic resonance imaging (MRI) images of a subject who is a target to be measured.

The biometric instrument for measurement and analysis 200 obtains a sensor output signal from the magnetic sensor 400, and outputs a result of analysis on the obtained signal, the result serving as information (brain function information) related to a function (a biological function) of the brain.

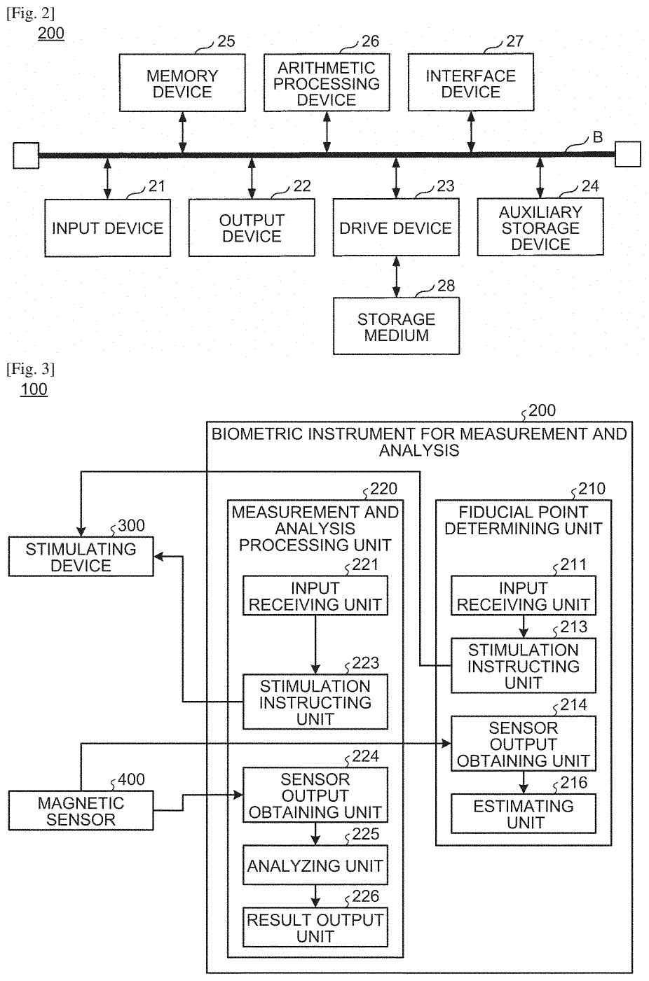

Described further hereinafter is the biometric instrument for measurement and analysis 200. FIG. 2 is a diagram illustrating an example of a hardware configuration of the biometric instrument for measurement and analysis 200.

The biometric instrument for measurement and analysis 200 is an information processing device including: an input device 21; an output device 22; a drive device 23; an auxiliary storage device 24; a memory device 25; an arithmetic processing device 26; and an interface device 27, which are connected to one another via a bus B.

The input device 21 is a device for inputting various types of information, and is realized by, for example, a keyboard and a pointing device. The output device 22 is for outputting various types of information, and is realized by, for example, a display. The interface device 27 includes a LAN card, and is used for connection to a network.

A biological function measuring and analyzing program is at least a part of various programs that control the biometric instrument for measurement and analysis 200. The biological function measuring and analyzing program is provided by, for example, being distributed through a storage medium 28, or being download from a network. Any of various types of storage media may be used as the storage medium 28 having the biological function measuring and analyzing program recorded therein, the various types of storage media including: a storage medium having information optically, electrically, or magnetically recorded therein, such as a CD-ROM, a flexible disk, or a magneto-optical disk; and a semiconductor memory having information electrically recorded therein, such as a ROM or a flash memory.

Furthermore, when the storage medium 28 having the biological function measuring and analyzing program recorded therein is set in the drive device 23, the biological function measuring and analyzing program is installed in the auxiliary storage device 24 via the drive device 23 from the storage medium 28. The biological function measuring and analyzing program downloaded from a network is installed in the auxiliary storage device 24 via the interface device 27.

The auxiliary storage device 24 stores therein the biological function measuring and analyzing program that has been installed, and also stores therein necessary files and data. The memory device 25 reads the biological function measuring and analyzing program from the auxiliary storage device 24 when the biometric instrument for measurement and analysis 200 is started, and stores therein the read biological function measuring and analyzing program. The arithmetic processing device 26 then realizes various types of processing described below, according to the biological function measuring and analyzing program stored in the memory device 25.

The stimulating device 300 is controlled by the biometric instrument for measurement and analysis 200. Specifically, the stimulating device 300 generates and outputs a stimulation to be given to the subject P, according to the control by the biometric instrument for measurement and analysis 200. Furthermore, the stimulating device 300 monitors a signal of a magnetic field or the like generated from the subject P, according to the control by the biometric instrument for measurement and analysis 200.

The stimulating device 300 may be, for example, an electrode provided on a belt. In this case, the stimulating device 300 is attached, for example, to an arm of the subject P, and an electric signal or a mechanical signal is given to the subject P as an electric stimulation or a locomotor stimulation.

Furthermore, the stimulating device 300 may be, for example, a display device or a sound output device. In this case, for example, the stimulating device 300 gives, as a visual stimulation (a light stimulation), a video displayed on the stimulating device 300, to the subject P, or gives, as an auditory stimulation (a sound stimulation), a sound output from the stimulating device 300, to the subject P.

Furthermore, in the biological function measuring and analyzing system 100, a signal generated from a neural activity of the brain of the subject P is detected by the magnetic sensor 400, but the detection is not limited to this example. The biological function measuring and analyzing system 100 preferably includes a sensor for detecting a signal generated from a neural activity of the brain; and this sensor is preferably minimally invasive, and more preferably non-invasive, for accurately measuring a biological function of the subject. Examples of this sensor include, in addition to a magnetic sensor, an electroencephalograph sensor (a voltage sensor), and an optical topography sensor (a near-infrared sensor).

Furthermore, the magnetic sensor 400 according to the embodiment may include different kinds of such sensors. However, in that case, operation of one of these sensors is required to not influence measurement by the other sensors. In particular, when a magnetic sensor is used as one of these sensors, since the magnetic sensor has a characteristic of being able to obtain a signal generated from a living body even if the magnetic sensor is not in contact with the living body, the state of attachment of the magnetic sensor does not influence the result of the measurement. Therefore, the magnetic sensor 400 is preferably used in the embodiment of the present invention.

Described next by reference to FIG. 3 are functions of the biometric instrument for measurement and analysis 200 according to the embodiment. FIG. 3 is a diagram illustrating functions of the biometric instrument for measurement and analysis 200.

The biometric instrument for measurement and analysis 200 has a fiducial point determining unit 210, and a measurement and analysis processing unit 220.

The fiducial point determining unit 210 and the measurement and analysis processing unit 220 are realized by the arithmetic processing device 26 reading and executing the biological function measuring and analyzing program that has been stored in the auxiliary storage device 24, the memory device 25, or the like.

The measurement and analysis processing unit 220 causes the stimulating device 300 to generate a stimulation, analyzes a sensor output signal detected by the magnetic sensor 400 correspondingly to this stimulation, and outputs a result of the analysis as a measurement result. The analysis of the sensor output signal includes: averaging signal waveforms; analyzing a signal waveform including an averaged waveform; analyzing a signal waveform by applying a frequency filter; analyzing a cerebral magnetic field including an orientation of a current dipole serving as a signal source; and analysis related to relations among plural signal sources, and brain functions are measured based on brain activity signals extracted by these kinds of analysis. The brain functions aimed to be measured include, for example: sensory functions, such as audition, vision, somatic sensation, olfaction, and gustation; the speech function; and the attentional functions.

The measurement and analysis processing unit 220 has an input receiving unit 221, a stimulation instructing unit 223, a sensor output obtaining unit 224, an analyzing unit 225, and a result output unit 226.

The input receiving unit 221 receives input of various types of information to the biometric instrument for measurement and analysis 200. Specifically, the input receiving unit 221 receives, for example, an operation that starts analysis of functions (biological functions) of a brain measured in the biological function measuring and analyzing system 100.

When the input receiving unit 221 receives the operation that starts the analysis of functions of the brain, the stimulation instructing unit 223 instructs the stimulating device 300 to generate a stimulation.

The sensor output obtaining unit 224 obtains a sensor output signal output from the magnetic sensor 400. Specifically, the sensor output obtaining unit 224 is connected to an output terminal or the like of the magnetic sensor 400, and obtains the sensor output signal output through the output terminal or the like.

The analyzing unit 225 performs analysis of the sensor output signal.

The result output unit 226 outputs a result of the analysis performed by the analyzing unit 225, the result serving as a result of measurement of functions of the brain.

Required in the biometric instrument for measurement and analysis 200 is relative positioning between brain anatomy data (an MRI image) obtained by the biological image measuring device 500 and brain function information obtained by the magnetic sensor 400 forming the brain function measuring device.

Therefore, before or after the magnetic sensor 400 measures brain function information, the fiducial point determining unit 210 gives a stimulation to the subject P by controlling the stimulating device 300, and induces brain activities at at least three fiducial points (FPs) that have been set in the cerebral parenchyma. The fiducial points are determined from brain activity signals based on the brain activities that have been induced. At the biological image measuring device 500 on the other hand, a measurer specifies coordinates of the fiducial points (FPs) on an MRI image. That is, positions of the FPs are able to be obtained in each coordinate system. Therefore, according to the present invention, positioning is performed by using fiducial points that have been set in the cerebral parenchyma, and thus positional accuracy in measurement of brain function information is able to be improved.

The fiducial point determining unit 210 has an input receiving unit 211, a stimulation instructing unit 213, a sensor output obtaining unit 214, and an estimating unit 216.

The input receiving unit 211 receives, for example, an operation that starts processing of determining positions of fiducial points in a brain measured in the biological function measuring and analyzing system 100.

When the input receiving unit 211 receives the operation that starts the processing of determining the positions of the fiducial points in the brain, the stimulation instructing unit 213 instructs the stimulating device 300 to generate a stimulation.

The sensor output obtaining unit 214 obtains a sensor output signal that is output from the magnetic sensor 400. Specifically, the sensor output obtaining unit 214 is connected to an output terminal or the like of the magnetic sensor 400, and obtains a sensor output signal output through the output terminal or the like.

Based on the sensor output signal obtained by the sensor output obtaining unit 214, the estimating unit 216 estimates and outputs parts of the cerebral parenchyma, the parts serving as the fiducial points. The stimulation instructing unit 213 may present the same stimulation a plural number of times. In this case, by averaging such sets of data, the estimating unit 216 is able to reduce a sensor signal (which will be called the noise) unrelated to the stimulation, and to take out only a brain activity signal reactive to the stimulation.

Described next is an example of fiducial point determination processing for determining fiducial points.

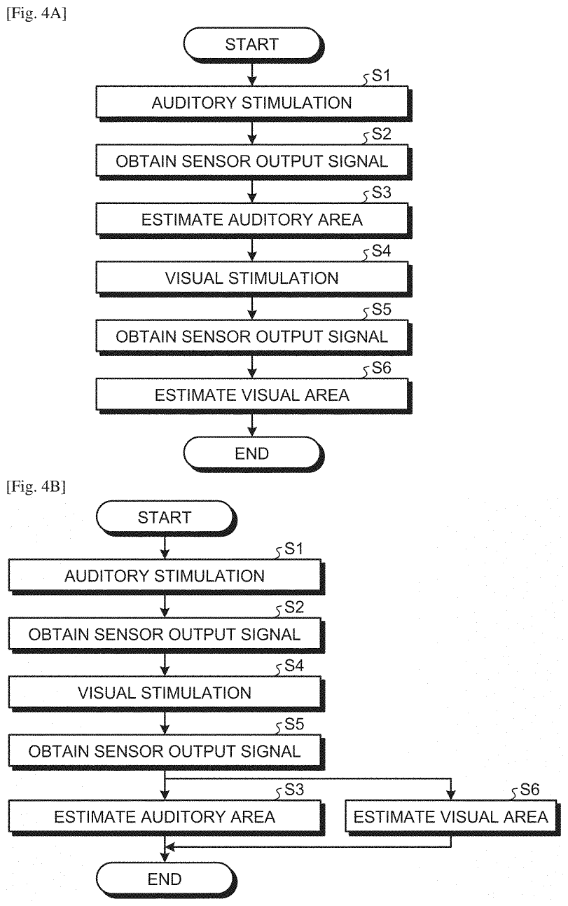

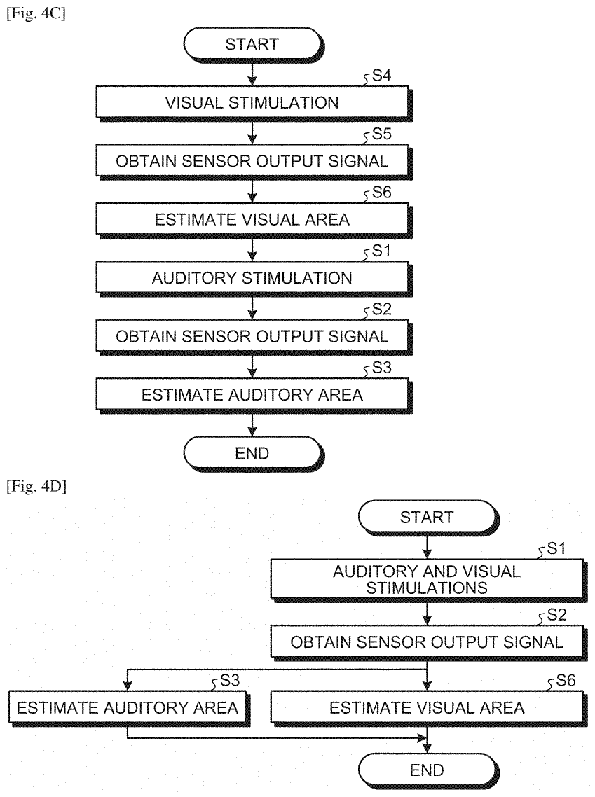

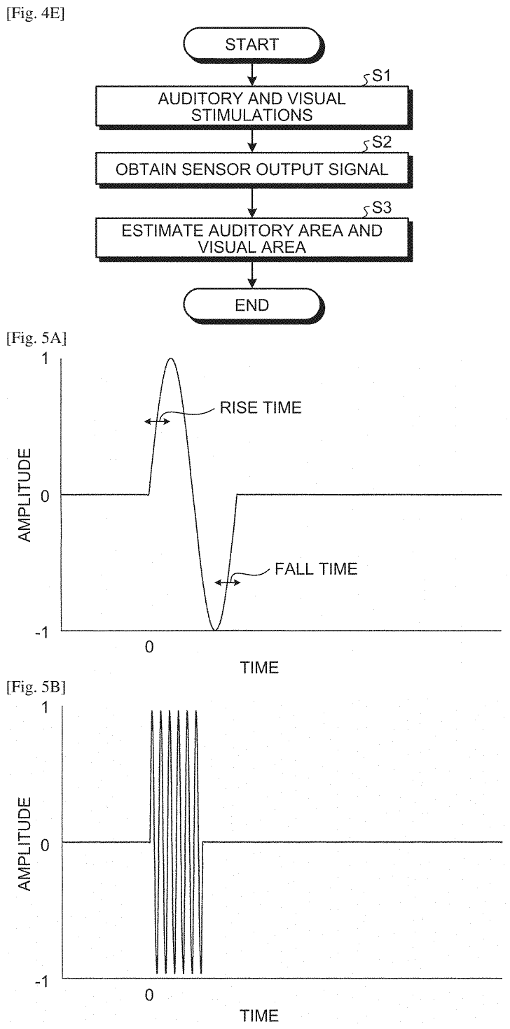

FIGS. 4A to 4E are flow charts schematically illustrating examples of flows of the fiducial point determination processing.

As illustrated in FIGS. 4A to 4E, the stimulation instructing unit 213 firstly provides an auditory stimulation (a sound stimulation) (Step S1). Subsequently, the sensor output obtaining unit 214 obtains a sensor output signal output from the magnetic sensor 400 (Step S2). Based on the sensor output signal obtained by the sensor output obtaining unit 214, the estimating unit 216 estimates positions of the left and right auditory areas of the subject (Step S3).

It is assumed herein that the auditory stimulation: has a waveform of a sine wave, a pulse wave, white noise, or the like; is able to be clearly separated from background noise; has a maximum sound volume that does not cause discomfort; and is in an audible frequency range.

FIGS. 5A and 5B are diagrams illustrating an example of a waveform of an audible sound stimulation. According to the waveform illustrated in FIG. 5A and FIG. 5B, the sound stimulation is an audible sound having a stimulation rise time and a stimulation fall time that are equal to or less than 100 milliseconds.

Subsequently, the stimulation instructing unit 213 provides a visual stimulation (a light stimulation) (Step S4). Subsequently, the sensor output obtaining unit 214 obtains a sensor output signal output from the magnetic sensor 400 (Step S5). Based on the sensor output signal obtained by the sensor output obtaining unit 214, the estimating unit 216 estimates a position of a visual area of the subject (Step S6).

The visual stimulation (light stimulation) may be of any color as long as the visual stimulation is in the range of visible light, and the visual stimulation is given as a flash stimulation that covers a field of view of a visual angle equal to or larger than one degree. Furthermore, the visual stimulation (light stimulation) may be given as a graphic pattern that is continuously inverted.

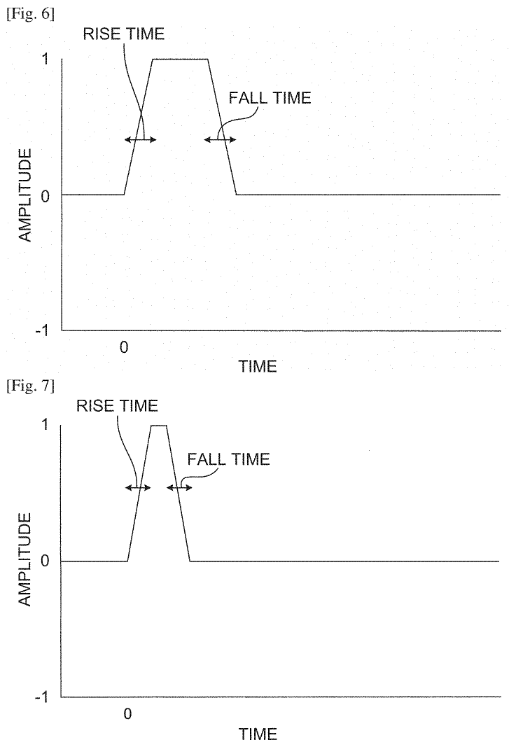

FIG. 6 is a diagram illustrating an example of a waveform of a visible light stimulation. According to the waveform illustrated in FIG. 6, the light stimulation is visible light having a stimulation rise time and a stimulation fall time that are equal to or less than 100 milliseconds.

Various modifications of the fiducial point determination processing according to the embodiment are possible. As illustrated in FIG. 4B, data obtained through Step S2 may be stored in a storage device, and the estimation of the positions of the left and right auditory areas of the subject (Step S3) and the estimation of the position of the visual area of the subject (Step S6) may be executed in parallel with each other. Furthermore, as illustrated in FIG. 4C, the execution sequence between the position estimation with the auditory stimulation (Steps S1 to S3) and the position estimation with the visual stimulation (Steps S4 and S5) may be changed. Moreover, as illustrated in FIG. 4D and FIG. 4E, the stimulation instructing unit 213 may simultaneously give the auditory stimulation (sound stimulation) and the visual stimulation (light stimulation). The processing is thereby able to be simplified. In FIG. 4D, the estimation of the positions of the left and right auditory areas of the subject (Step S3) and the estimation of the position of the visual area of the subject (Step S6) are executed in parallel with each other. In FIG. 4E, on the other hand, at Step S3, in addition to the estimation of the positions of the left and right auditory areas of the subject, the estimation of the position of the visual area of the subject is also performed.

According to this embodiment, the stimulation instructing unit 213 provides an auditory stimulation (a sound stimulation) and a visual stimulation (a light stimulation), but not being limited thereto, the stimulation instructing unit 213 may provide an electric stimulation or the like. The electric stimulation directly stimulates nerves, and induces a brain activity in a somatosensory area. The electric stimulation has a waveform of a sine wave, a pulse wave, white noise, or the like, and has a current equal to or less than 100 mA.

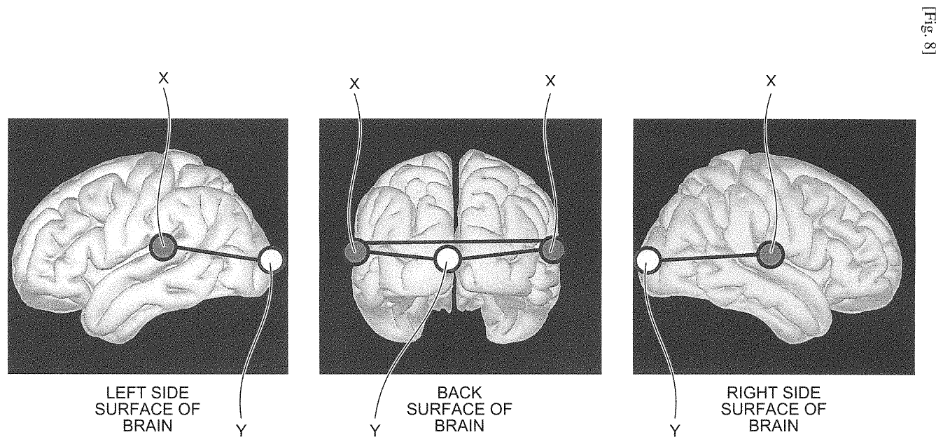

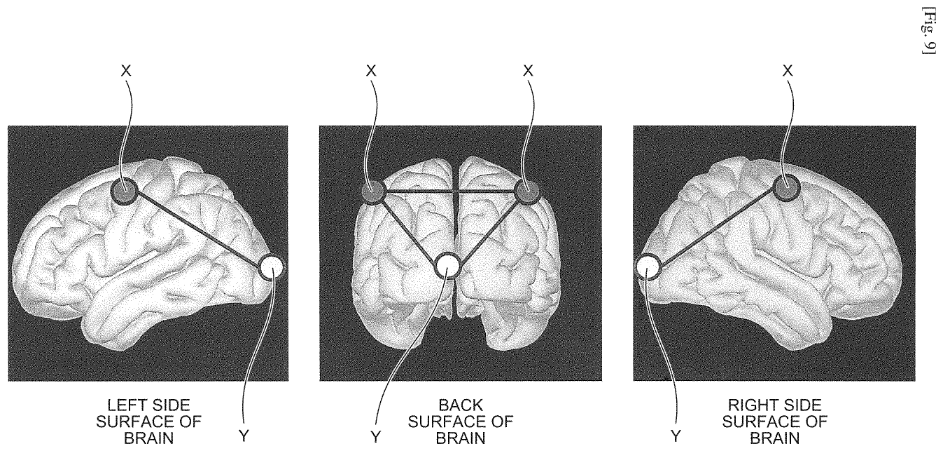

FIG. 7 is a diagram illustrating an example of a waveform of the electric stimulation.

According to the waveform illustrated in FIG. 7, the electric stimulation is an electric stimulation having a stimulation rise time and a stimulation fall time that are equal to or less than 20 milliseconds.

Furthermore, instead of an auditory stimulation (a sound stimulation), a visual stimulation (a light stimulation), or an electric stimulation; a locomotor stimulation may be used. In this case, the stimulation instructing unit 213 presents a sound or a video having content instructing the subject to make a movement, such as holding the subject's own hand. This movement of the subject herself induces a brain activity in a motor area in the brain of the subject, and thus the motor area is able to be estimated.

If the same stimulation is presented for a plural number of times and averaging is performed, a stimulation that allows the level of a brain activity signal reactive to the stimulation to be large as compared to the noise is preferably used to decrease the addition frequency and to accurately perform positioning in a short period of time. A spontaneous movement of the subject, for example, may serve as such a stimulation, and a brain activity signal in the motor area may thus be used.

Furthermore, if the stimulation instructing unit 213 uses a stimulation short in time (latent time) from presentation of the stimulation to generation of a brain activity signal, the number of measurements executable in a predetermined time period is able to be increased, the number of additions per unit time is able to be increased, and thus accurate positioning is able to be performed. An electric stimulation, for example, may serve as such a stimulation, and a brain activity signal in the somatosensory area may thus be used.

Parts where brain activities occur due to plural stimulations provided by the stimulation instructing unit 213 are preferably positioned in areas that are separated from one another. This is because the accuracy of positioning is improved when such parts where the brain activities occur are made the fiducial points.

In particular, when a part where a brain activity occurs due to at least one stimulation appears in the left hemisphere of the brain, and a part where a brain activity occurs due to the same stimulation or at least another stimulation appears in the right hemisphere of the brain; positioning is able to be performed by using an area that is across both the left and right hemispheres of the brain.

The stimulation instructing unit 213 according to the embodiment preferably gives an auditory stimulation (a sound stimulation) to both of the ears so that brain activity signals in both the left and right auditory areas are able to be used in the estimation.

Furthermore, if one of the stimulations is a visual stimulation (a light stimulation) like in the embodiment, the visual area positioned at a back end of the brain is a part where a brain activity occurs; when this visual stimulation is combined with another stimulation, such as a sound stimulation (auditory areas), a locomotor stimulation (a motor area), or an electric stimulation (a somatosensory area), the fiducial points will be at positions separate from one another; and thus one of the stimulations is preferably a visual stimulation (a light stimulation).

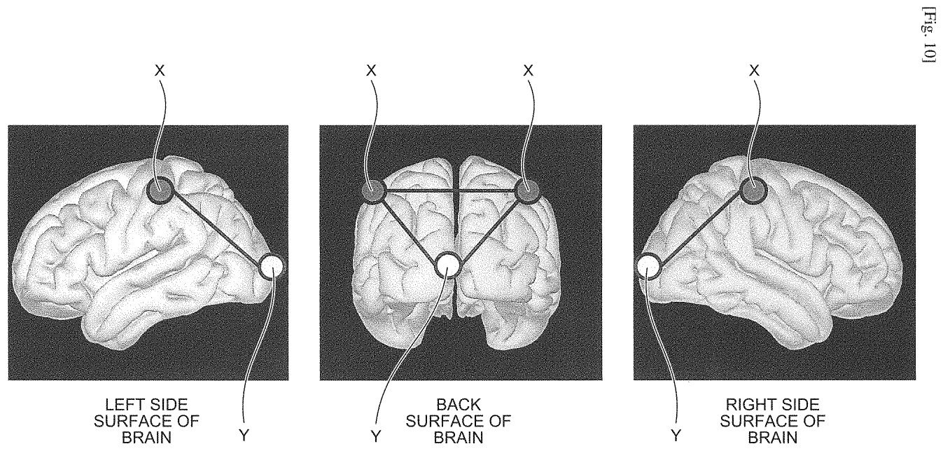

FIG. 8 is a diagram illustrating an example of parts where brain activities occur due to a light stimulation and a sound stimulation. In the example of FIG. 8, the estimating unit 216 estimates two points (X in FIG. 8) that are parts where brain activities occur due to a sound stimulation and a point (Y in FIG. 8) that is a part where a brain activity occurs due to a light stimulation, and determines these points as fiducial points.

FIG. 9 is a diagram illustrating an example of parts where brain activities occur due to a light stimulation and a locomotor stimulation. In the example of FIG. 9, the estimating unit 216 estimates two points (X in FIG. 9) that are parts where brain activities occur due to a locomotor stimulation and a point (Y in FIG. 9) that is a part where a brain activity occurs due to a light stimulation, and determines these points as fiducial points.

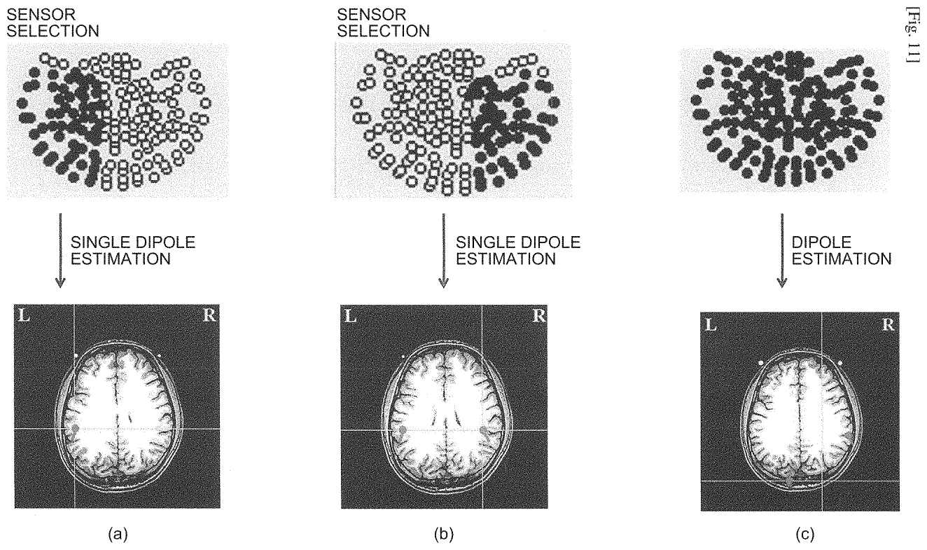

FIG. 10 is a diagram illustrating an example of parts where brain activities occur due to a light stimulation and an electric stimulation. In the example of FIG. 10, the estimating unit 216 estimates two points (X in FIG. 10) that are parts where brain activities occur due to an electric stimulation and a point (Y in FIG. 10) that is a part where a brain activity occurs due to a light stimulation, and determines these points as fiducial points.

Furthermore, by combining an auditory stimulation (a sound stimulation) and a visual stimulation (a light stimulation) together like in this embodiment, left, right and back ampullas of the brain are able to be captured, and accuracy of positioning is able to be improved.

Furthermore, when a locomotor stimulation and a visual stimulation (a light stimulation) are combined together, positioning is able to be performed in a number of times of addition less than that for an auditory stimulation (a sound stimulation).

Furthermore, when an electric stimulation and a visual stimulation (a light stimulation) are combined together, positioning is able to be performed in an execution time period shorter than that for an auditory stimulation (a sound stimulation).

Accordingly, the fiducial point determining unit 210 according to the embodiment determines positions of the left and right auditory areas and a position of the visual area as fiducial points, and associates these three points with brain anatomy data (an MRI image).

Briefly described below is a technique for estimating an active part of a brain in the analyzing unit 225 of the measurement and analysis processing unit 220. Examples of the technique for estimating an active part of a brain include a dipole estimation method, and a spatial filtering method. The analyzing unit 225 of the measurement and analysis processing unit 220 according to the embodiment estimates an active part of a brain by a dipole estimation method using some sensors of the magnetic sensor 400.

FIG. 11 is a diagram illustrating examples of dipole estimation techniques for active parts of a brain. As illustrated in FIG. 11, in this example, an active part of a brain is estimated by a dipole estimation method using some sensors of the magnetic sensor 400 in a case where an audible sound is given to a subject and brain activities in auditory areas (Heschl's convolutions) are induced.

It has been known that a stimulation with an audible sound induces brain activities in

Heschl's convolutions of both the left and right brain hemispheres of a subject. In this case, as illustrated in FIG. 11(a), firstly, single dipole estimation is performed by use of some sensors of the magnetic sensor 400, the some sensors being positioned at the left hemisphere side. Thereafter, subsequently, as illustrated in FIG. 11(b), single dipole estimation is performed by use of some sensors of the magnetic sensor 400, the some sensors being positioned at the right hemisphere side. Two dipole positions corresponding to the brain activities in the Heschl's convolutions on both sides are thereby able to be estimated.

Enabled by this procedure is position estimation that is more accurate than, for example, when two dipole positions are simultaneously estimated (see FIG. 11(c)) by use of all of the sensors in the left and right brain hemispheres. In this example, a GOF of 95% or more is able to be achieved. Accordingly, when a stimulation that respectively causes brain activities in parts that are present in left and right brain hemispheres is given, by performing estimation of active parts of the brain by the dipole estimation method using some sensors of the magnetic sensor 400, positions of parts where brain activities occur are able to be identified accurately.

Furthermore, signal source estimation in a case where activities in plural parts of a brain have been induced is also able to be performed by use of a spatial filtering method. For example, when an auditory stimulation (a sound stimulation) and a visual stimulation (a light stimulation) are given, brain activities in three parts, the left and right auditory areas and the visual area, will be induced. In this case, each active part of the brain is able to be estimated accurately by use of a spatial filtering method. A flow of fiducial point determination processing in this case will be like the one in the flow chart illustrated in FIG. 4E.

As described above, according to the embodiment, a stimulation is given to a subject, the stimulation enabling at least three points in brain anatomy data (for example, an MRI image) obtained by the biological image measuring device 500 to be determined; coordinates of three or more signal sources obtained are fitted to the brain anatomy data; and positions in the brain where brain function information is being measured by the magnetic sensor 400 forming the brain function measuring device are thereby determined. Since the brain function information and the brain anatomy data are thereby associated with each other by using, instead of information on positions on a surface of the head, information on positions in the cerebral parenchyma of a subject; positional accuracy of brain mapping is able to be improved significantly.

The brain anatomy data according to the present invention may be not an MRI image of a subject himself. For example, brain anatomy data of another person, or brain anatomy data of a standard brain may be used, the brain anatomy data of the standard brain having been subjected to affine transformation such that fiducial points set for these brain anatomy data match fiducial points determined by the fiducial point determining unit 210.

### REFERENCE SIGNS LIST

100 System

200 Brain function measuring device

300 Stimulating device

213 Stimulation instructing unit

216 Estimating unit

400 Sensor

### CITATION LIST

### Patent Literature

PTL 1: Japanese Patent No. 5967605

1. A measuring device comprising:
a stimulation instructing unit configured to instruct a stimulating device configured to give a plurality of stimulations to a brain of a subject who is a target to be measured, to generate a plurality of stimulations for inducing brain activities in at least three areas separated from one another in the brain; and
an estimating unit configured to estimate, based on sensor output signals output from sensors configured to measure a brain activity signal of the subject , parts where brain activities occur due to the plurality of stimulations, the parts corresponding to the at least three areas, wherein
the measuring device associates the estimated parts where the brain activities occur, with points corresponding to the at least three areas in brain anatomy data.

2. The measuring device according to claim 1, wherein in the stimulation instructing unit, the plurality of stimulations include a sound stimulation.

3. The measuring device according to claim 2, wherein the sound stimulation is an audible sound having a stimulation rise time and a stimulation fall time that are equal to or less than 100 milliseconds.

4. The measuring device according to claim 2, wherein the estimating unit is configured to estimate parts where brain activities occur in both left and right hemispheres by a dipole method using some of the sensors.

5. The measuring device according to claim 1, wherein in the stimulation instructing unit, the plurality of stimulations include a locomotor stimulation.

6. The measuring device according to claim 1, wherein in the stimulation instructing unit, the plurality of stimulations include an electric stimulation.

7. The measuring device according to claim 6, wherein the electric stimulation is an electric stimulation having a stimulation rise time and a stimulation fall time that are equal to or less than 20 milliseconds.

8. The measuring device according claim 2, wherein in the stimulation instructing unit, the plurality of stimulations include a light stimulation.

9. The measuring device according to claim 8, wherein the light stimulation is visible light having a stimulation rise time and a stimulation fall time that are equal to or less than 100 milliseconds.

10. The measuring device according to claim 1, wherein the plurality of stimulations include a sound stimulation and a light stimulation, and the stimulation instructing unit is configured to simultaneously give instructions for generating the plurality of stimulations.

11. The measuring device according to claim 10, wherein the estimating unit is configured to estimate a part where a brain activity occurs by a spatial filtering method.

12. A system comprising:
a stimulating device configured to generate and output a plurality of stimulations;
sensors configured to measure a brain function of a subject who is a target to be measured; and
the measuring device according to claim 1.

13. (canceled)

14. The system according to claim 1, wherein the brain anatomy data are based on a standard brain.

15. A method comprising:
instructing a stimulating device configured to give a plurality of stimulations to a brain of a subject who is a target to be measured, to generate a plurality of stimulations for inducing brain activities in at least three areas separated from one another in the brain;
estimating, based on sensor output signals output from sensors configured to measure a brain activity signal of the subject, parts where brain activities occur due to the plurality of stimulations, the parts corresponding to the at least three areas; and
associating the estimated parts where the brain activities occur, with points corresponding to the at least three areas in brain anatomy data.
